(19) United States
(12) Patent Application Publication
(10) Pub. No.: US 2004/0236233 A1
(43) Pub. Date: Nov. 25, 2004

(54) Information-gathering device and pulse meter

(75) Inventors: Tsukasa Kosuda, Matsumato-shi (JP); Yutaka Kawafune, Matsumoto-shi (JP); Makoto Zakoji, Shiojiri-shi (JP); Shoichi Nagao, Okaya-shi (JP)

Correspondence Address: SHINJYU GLOBAL IP COUNSELORS, LLP, 1233 20TH STREET, NW, SUITE 700, WASHINGTON, DC, 20036-2680, US

(73) Assignee: Seiko Epson Corporation, Shinjuku-ku (JP)

(21) Appl. No.: 10/803,088

(22) Filed: Mar. 18, 2004

(30) Foreign Application Priority Data
Mar. 19, 2003 (JP) 2003-075838

(51) Int. Cl. A61B 5/02

(52) U.S. Cl. 600/485

(57) ABSTRACT

In a pulse meter, a motion detector detects motion components generated along with changes in the shape of a mounting area on the body, and outputs a motion detection signal to a transmitter. A pulse wave detector detects pulse wave components and outputs a pulse wave detection signal to the transmitter. A pulse rate calculator calculates the pulse rate on the basis of the motion detection signal and the pulse wave detection signal. Motion components can be removed from pulse wave components and the pulse rate can be accurately calculated even when motion components with low acceleration are generated.

### BACKGROUND OF THE INVENTION

1. Field of the Invention

The present invention relates to an information-gathering device and a pulse meter. More specifically, the present invention relates to an information-gathering device and a pulse meter mounted on part of the body and used to measure the pulse during walking or running.

2. Background Information

Pulse meters mounted on part of the body and designed for measuring pulse during walking or running are conventionally known. For example, a wristwatch-type pulse meter is disclosed in Japanese Patent No. 2816944, which is hereby incorporated by reference. The pulse meter disclosed in JP2816944 employs a configuration wherein the frequency components corresponding to all the harmonic components of a motion signal detected by an acceleration sensor are removed from the frequency analysis results of a pulse wave signal on the basis of the frequency analysis results of the motion signal. Further, the frequency components having the maximum power are extracted from among the frequency analysis results of the pulse wave signal from which the harmonic components of the motion signal have been removed. Finally, the pulse rate is calculated based on the extracted frequency components. In the above-mentioned conventional pulse meter, the motion components are detected with an acceleration sensor, and problems have therefore been encountered in the sense that the motion components cannot be detected in operations with low acceleration, and the correct pulse wave components cannot be extracted even when there is a marked effect on the pulse wave signal.

Clenching and unclenching the hand is an example of such an operation with low acceleration in a wristwatch-type pulse meter. The diameter of the wrist changes by several millimeters when the hand is clenched and unclenched. This affect is pronounced in pulse wave components, but does not appear in motion components. Therefore, problems have been encountered in that sometimes the pulse wave components cannot be accurately extracted and the correct pulse cannot be accurately measured.

In view of the above, it will be apparent to those skilled in the art from this disclosure that there exists a need for an improved information-gathering device and pulse meter. This invention addresses this need in the art as well as other needs, which will become apparent to those skilled in the art from this disclosure.

### SUMMARY OF THE INVENTION

An object of the present invention is to provide an information-gathering device and a pulse meter that can accurately calculate the pulse rate even when motion components with low acceleration are generated. The pulse meter relating to the present invention is mounted on the body. In this pulse meter, a motion detector detects motion components generated along with changes in the shape of the mounting area of the body, and outputs a motion detection signal to a transmitter. A pulse wave detector detects pulse wave components and outputs a pulse wave detection signal to the transmitter. A pulse rate calculator calculates the pulse rate on the basis of the motion detection signal and the pulse wave detection signal.

According to the present invention, when pulse wave components are extracted from the frequency analysis results of both the pulse wave detector and the motion detector, the motion components are removed from the pulse wave components to calculate accurately the pulse rate, and the precision of pulse detection can be improved even when motion components with low acceleration are generated. In this case, the motion detector may be configured from first and second motion detectors. The first motion detector detects motion components generated along with changes in the shape of the mounting area of the body and outputs a first motion detection signal. The second motion detector detects motion components generated along with movement of the body and outputs a second motion detection signal.

These and other objects, features, aspects, and advantages of the present invention will become apparent to those skilled in the art from the following detailed description, which, taken in conjunction with the annexed drawings, discloses a preferred embodiment of the present invention.

### BRIEF DESCRIPTION OF THE DRAWINGS

Referring now to the accompanying drawings which form a part of this original disclosure:

FIG. 1 is a view of a schematic structural diagram of a pulse measurement system in accordance with a first preferred embodiment of the present invention;

FIG. 2 is a view of an explanatory diagram of a mounted sensor module of the pulse measurement system;

FIG. 3 is a view of a schematic structural block diagram of the sensor module and a portable device of the pulse measurement system;

FIG. 4 is a schematic cross-sectional view of the sensor module;

FIG. 5 is a view of an explanatory diagram of frequency analysis results of pulse wave detection data received by a microprocessor unit (the MPU) of the pulse measurement system;

FIG. 6 is a view of an explanatory diagram of frequency analysis results of motion detection data received by the MPU;

FIG. 7 is a view of an explanatory diagram of differential data, which are the difference between the pulse wave detection data analyzed for frequency and the motion detection data analyzed for frequency;

FIG. 8 is a view of an explanatory diagram of frequency analysis results of differential data;

FIG. 9 is a view of an explanatory diagram of frequency analysis results of pulse wave detection data;

FIG. 10 is a view of an explanatory diagram of frequency analysis results of motion detection data;

FIG. 11 is a view of an explanatory diagram of differential data, which are the difference between the pulse wave detection data analyzed for frequency and the motion detection data analyzed for frequency;

FIG. 12 is a view of a schematic structural block diagram illustrating one example of an adaptive filter of the pulse measurement system;

FIG. 13 is a view of a graph of a chronological arrangement of one example of pulse wave detection data;

FIG. 14 is a view of a graph in which motion detection data correlated with the pulse wave detection data in FIG. 13 are chronologically arranged along the same time axis;

FIG. 15 is a view of a graph of a chronological arrangement of differential data obtained by applying an adaptive filter to the pulse wave detection data in FIG. 13 and the motion detection data in FIG. 14;

FIG. 16 is a view of a frequency analysis results obtained by subjecting the differential data in FIG. 15 to FFT;

FIG. 17 is a view of a schematic structural block diagram of a sensor module and a portable device of the pulse measurement system in accordance with a second preferred embodiment of the present invention;

FIG. 18 is a view of a schematic cross-sectional view of a sensor module of the pulse measurement system in accordance with the second embodiment;

FIG. 19 is a view of a schematic structural block diagram of one example of an adaptive filter of the pulse measurement system in accordance with the second embodiment;

FIG. 20 is a view of a schematic structural block diagram of an alternate example of an adaptive filter of the pulse measurement system in accordance with the second embodiment;

FIG. 21 is a view of an explanatory diagram of an application of the pulse measurement system;

FIG. 22 is an elevational view illustrating the configuration of a power generation device of the pulse measurement system;

FIG. 23 is a schematic cross-sectional side view of the power generation device as seen from the direction indicated by the arrow (????) in FIG. 22;

FIG. 24 is a view of a schematic structural diagram of a voltage control circuit of the pulse measurement system;

FIG. 25 is a view of an explanatory diagram of a modification of a rotor of the pulse measurement system; and

FIG. 26 is a view of an explanatory diagram illustrating the motion detection sensor mounted on the same axis on the other side of the wrist.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

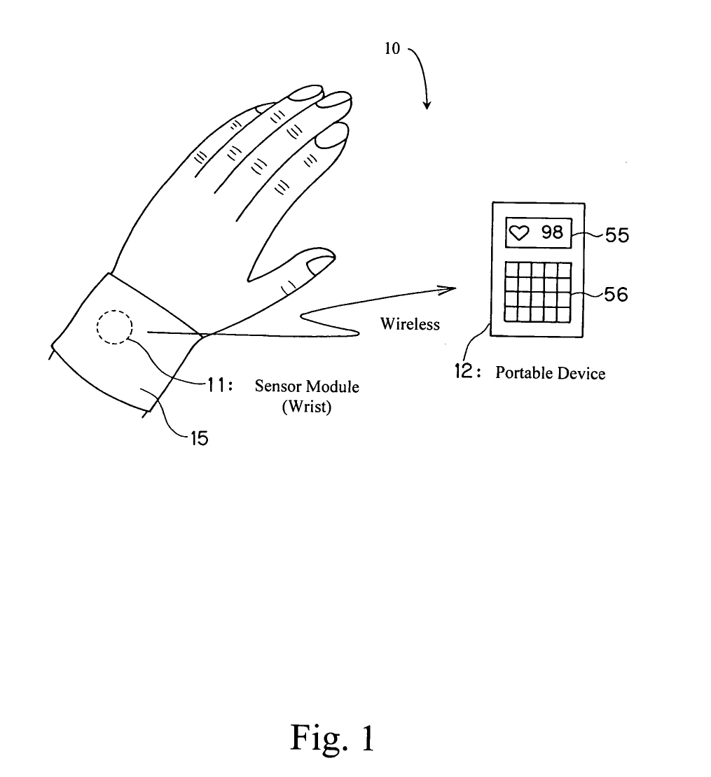

Initially, the first embodiment of the present invention will be described with reference to FIGS. 1 through 8. FIG. 1 is a view of a schematic structural diagram of a pulse measurement system (information-gathering device) 10 in accordance with a first preferred embodiment of the present invention. In general terms, the pulse measurement system 10 is configured from a sensor module 11 mounted on the arm of the user, and a PDA (Personal Digital Assistant), a portable phone, or the like, and has a portable device 12 connected to the sensor module 11 via wireless transmission. The pulse measurement system 10 according to the first embodiment makes it possible to detect and to register accurately motion components generated from deformations in the mounting area typified by deformations in the wrist (increase and decrease in wrist diameter) due to the clenching and unclenching of the hand. Therefore, the motion components can be accurately removed from the collected data, making it possible to detect accurately pulse wave components, and hence to measure accurately the pulse rate.

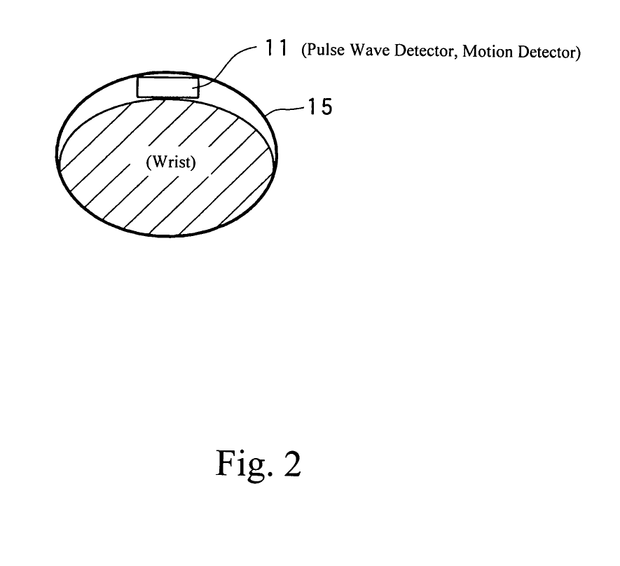

FIG. 2 is a view of an explanatory diagram of a sensor module 11 of the pulse measurement system that has been mounted. As can be seen in FIGS. 1 and 2, the sensor module 11 is mounted pressed against the wrist with a supporter 15. The supporter 15 is elastic and is fitted to the wrist by inserting the wrist therethrough, which presses the sensor module 11 to the back of the wrist.

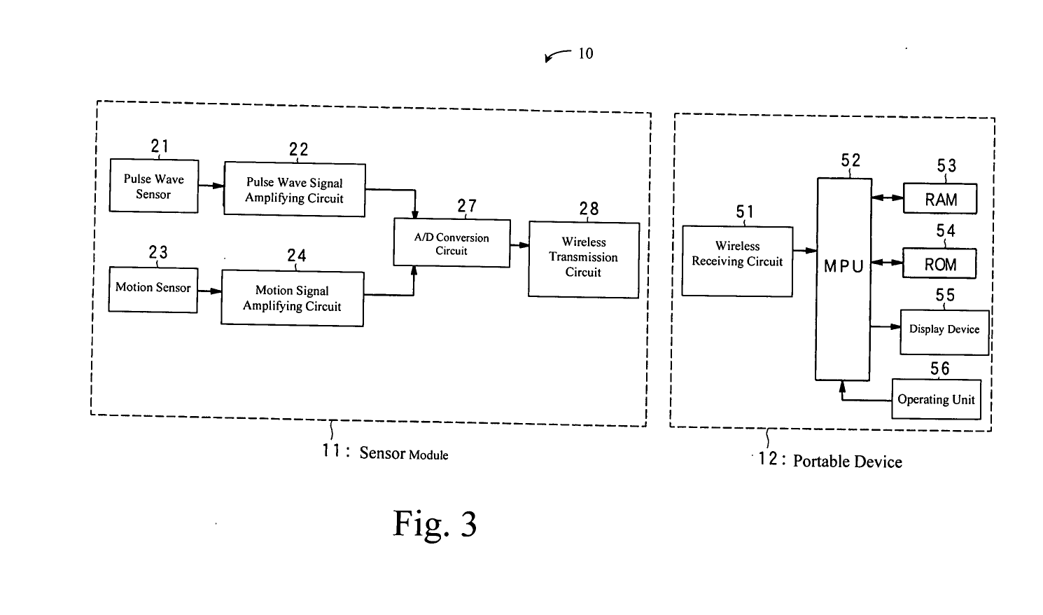

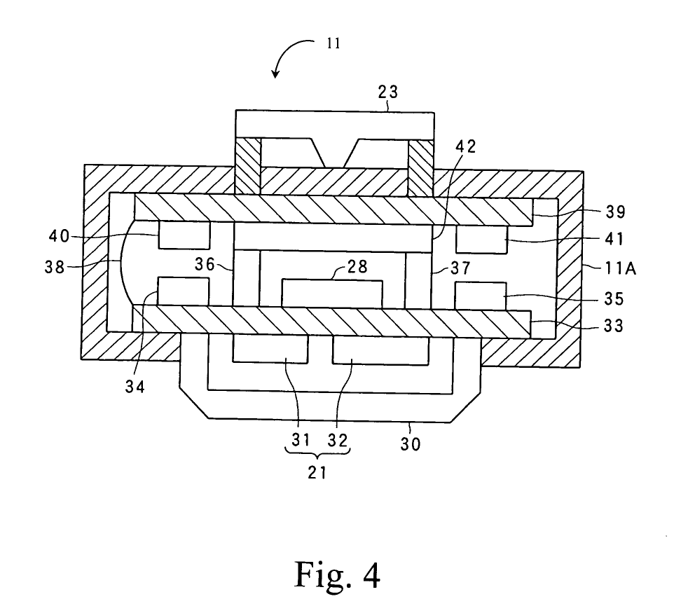

FIG. 3 is a view of a schematic structural block diagram of the sensor module 11 and the portable device 12. In general terms, the sensor module 11 has a pulse wave sensor (pulse wave detector) 21, a pulse wave signal amplifying circuit 22, a motion sensor (motion detector) 23, a motion signal amplifying circuit 24, an A/D conversion circuit 27, and a wireless transmission circuit (transmitter) 28. As shown in FIG. 4, the pulse wave sensor 21 has an LED (Light Emitting Diode) 31 and a PD (Photo Detector) 32. Referring again to FIG. 3, further, the pulse wave sensor 21 presents the pulse wave signal amplifying circuit 22 with a pulse wave detection signal that corresponds to the pulsating flow due to the heart rate of blood flowing through the blood vessels. The pulse wave signal amplifying circuit 22 amplifies the inputted pulse wave detection signal at a specific rate of amplification and outputs the result as an amplified pulse wave signal to the A/D conversion circuit 27. The motion sensor 23 detects changes in the shape of the mounting area of the sensor module 11, or, specifically, changes in the wrist diameter due to the clenching and unclenching of the hand, and outputs a motion detection signal to the motion signal amplifying circuit 24. In this case, the motion sensor 23 can be configured from a load sensor, a pressure sensor, a displacement sensor, or the like, but an example in which a load sensor is used is described below. The motion signal amplifying circuit 24 amplifies the inputted motion detection signal at a specific rate of amplification, and outputs the result as an amplified motion signal to the A/D conversion circuit 27. The A/D conversion circuit 27 performs analog/digital conversion on the inputted amplified pulse wave signal, and outputs the result as pulse wave detection data to the wireless transmission circuit 28. The A/D conversion circuit 27 also performs analog/digital conversion on the inputted amplified motion signal, and outputs the result as motion detection data to the wireless transmission circuit 28. The wireless transmission circuit 28 modulates the carrier wave on the basis of the inputted pulse wave detection data and motion detection data, and transmits the result to the portable device 12.

The mechanical configuration of the sensor module 11 will now be described. FIG. 4 is a schematic cross-sectional view of the sensor module 11. The sensor module 11 is designed so that the lower side in FIG. 4 is pressed against the arm of the user. In other words, the cover glass 30 side faces the arm of the user. Therefore, the LED 31 and PD 32 constituting the pulse wave sensor 21 are aligned on a first board 33 in a state protected by a cover glass 30 on the lower side of a casing 11A of the sensor module 11. The first board 33 is supported by the casing 11A. The wireless transmission circuit 28, circuit elements 34 and 35, and battery supports 36 and 37 are aligned on the upper side of the first board 33.

A second board 39 is connected to the first board 33 via a flexible wiring board 38. This second board 39 is supported by the casing 11A. Circuit elements 40 and 41 are aligned on the lower side of the second board 39. Furthermore, a power source 42 is brought into contact while supported by the battery supports 36 and 37. In addition, the motion sensor 23 is supported on the upper side of the casing 11A, and the motion sensor 23 is electrically connected to the second board 39.

Referring now to FIG. 3, the configuration of the portable device 12 will now be described. In general terms, the portable device 12 has a wireless receiving circuit (receiver) 51, an MPU (pulse rate calculator) 52, a RAM 53, a ROM 54, a display device 55, and an operating unit 56. The wireless receiving circuit 51 receives the pulse wave detection data and motion detection data transmitted from the wireless transmission circuit 28 of the sensor module 11, and outputs it to the MPU 52. The MPU 52 controls the portable device 12. The RAM 53 temporarily stores various data. The ROM 54 stores the control programs and the like used by the MPU 52 in advance. The display device 55 has a liquid crystal display or the like, and displays pulse rate data and other such various data under the control of the MPU 52. The operating unit 56 preferably has operating buttons and other such operating elements, and is used to input data, commands, and the like.

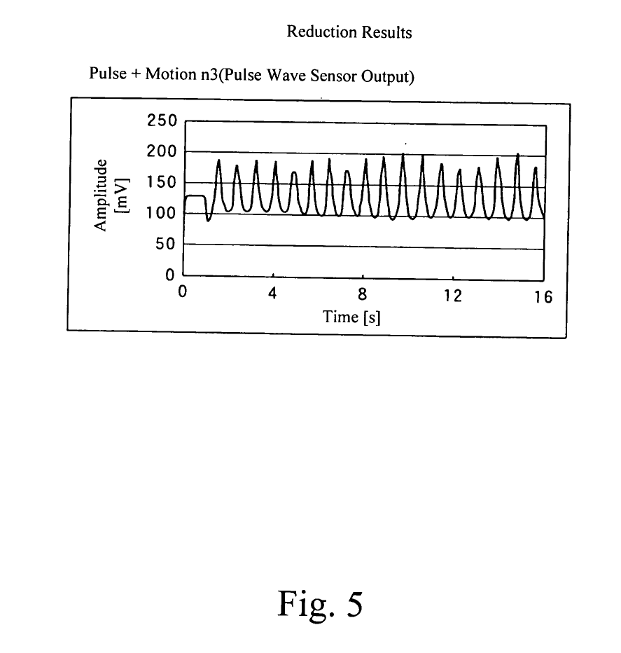

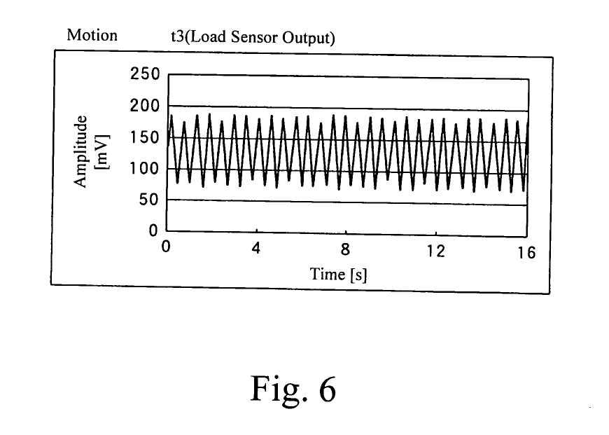

The pulse rate calculation processing performed in the MPU 52 that has received the pulse wave detection data and motion detection data will now be described. FIG. 5 is a view of an explanatory diagram of the frequency analysis results of pulse wave detection data received by the MPU 52. FIG. 6 is a view of an explanatory diagram of the frequency analysis results of motion detection data received by the MPU 52. First, the MPU 52 receives pulse wave detection data and motion detection data via the wireless receiving circuit 51, and stores the data sequentially in the RAM 53. When a specific volume of data is stored in the RAM 53, the MPU 52 then sequentially reads the pulse wave detection data and the motion detection data stored in the RAM 53, subjects the results to FFT, and performs frequency analysis.

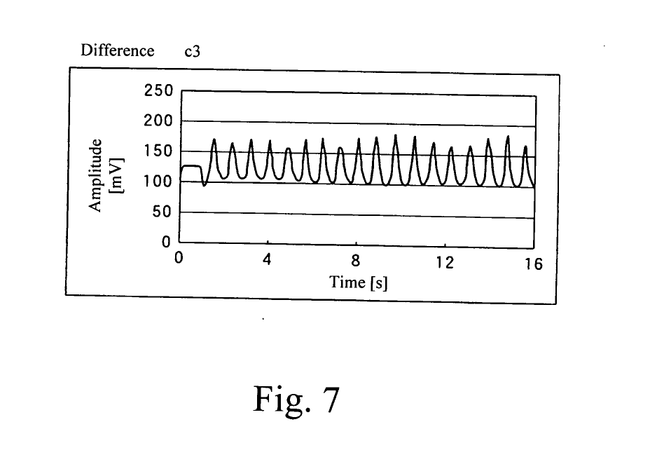

FIG. 7 is a view of an explanatory diagram of differential data, which is the difference between the pulse wave detection data analyzed for frequency and the motion detection data analyzed for frequency. The MPU 52 compares the pulse wave detection data analyzed for frequency and the motion detection data analyzed for frequency, and determines the difference between their frequency components to create differential data.

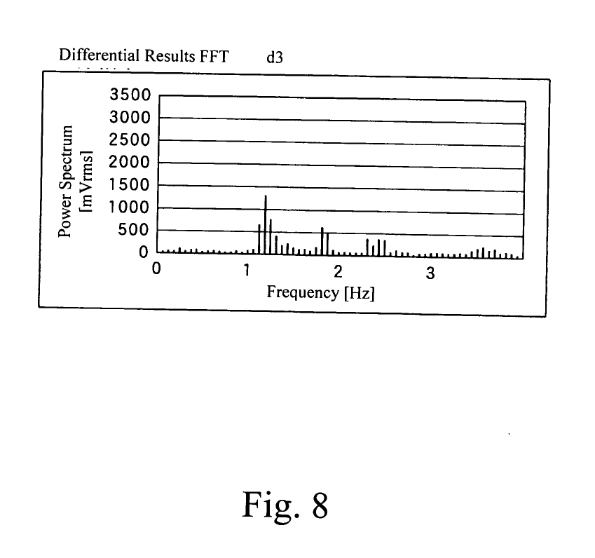

FIG. 8 is a view of an explanatory diagram of the frequency analysis results of the differential data. Thus, the frequency analysis results of the resulting differential data constitute data in which the motion components originating in the deformation of the wrist due to the clenching and unclenching of the hand, for example, are substantially removed from the output signal (pulse wave components+motion components) of the pulse wave sensor, specifically, pulse wave data that primarily correspond to the pulse wave components. Furthermore, the MPU 52 calculates the pulse rate from the frequency on the assumption that the maximum frequency components from the resulting pulse wave data constitute the pulse spectrum. The MPU 52 then displays the pulse rate on the display device 55.

As described above, according to the first embodiment, it is possible to detect and to register accurately motion components generated from deformations in the mounting area typified by deformations in the wrist (increase and decrease in wrist diameter) due to the clenching and unclenching of the hand. Therefore, the motion components originating in deformations in the mounting area can be accurately removed, making it possible to detect accurately pulse wave components, and hence to measure accurately the pulse rate.

### First Modification of the First Embodiment

The first embodiment described above uses a configuration wherein the MPU 52 subtracts pressure detection data from pulse wave detection data prior to frequency analysis (FFT) and calculates differential data, whereas the first modification of the first embodiment uses a configuration wherein the MPU 52 calculates the differential data after performing frequency analysis on the pulse wave detection data and motion detection data. Otherwise, the first modification of the first embodiment has the same configuration as the first embodiment. Therefore, the main differences of the first modification of the first embodiment from the configuration of the first embodiment will now be described.

In the first modification of the first embodiment, the MPU 52 performs frequency analysis (FFT) on both the pulse wave detection data and the motion detection data stored in the RAM 53. Next, the MPU 52 determines the differential data, which is the difference between the pulse wave detection data analyzed for frequency and the motion detection data analyzed for frequency. The harmonic components of the pulse wave are then extracted from the resulting differential data, and the pulse rate is calculated from the frequency.

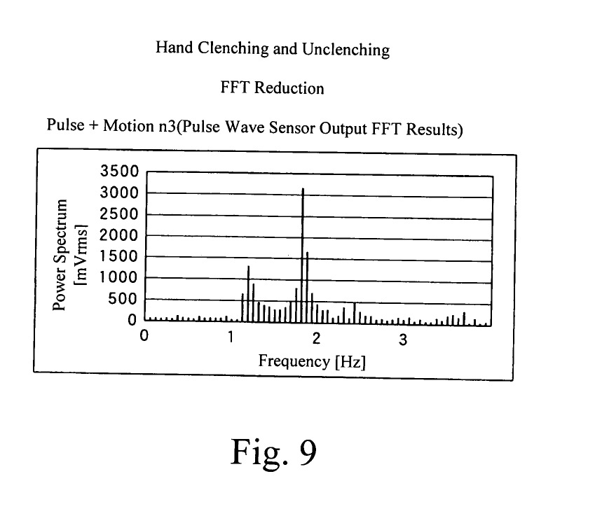

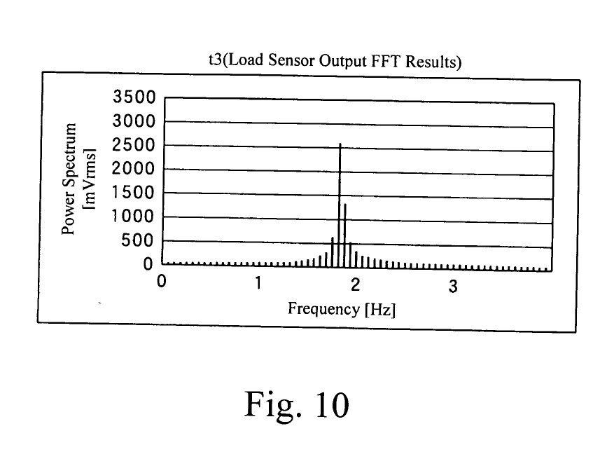

A more specific pulse rate calculation process will now be described. FIG. 9 is a view of an explanatory diagram of the frequency analysis results of pulse wave detection data. FIG. 10 is a view of an explanatory diagram of the frequency analysis results of motion detection data. First, the MPU 52 sequentially reads the pulse wave detection data and the motion detection data stored in the RAM 53, subjects the results to FFT, and performs frequency analysis.

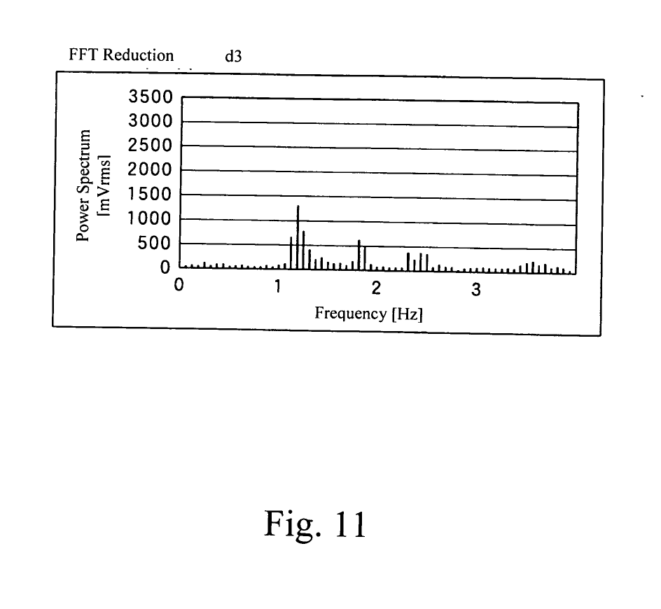

FIG. 11 is a view of an explanatory diagram of differential data, which is the difference between the pulse wave detection data analyzed for frequency and the motion detection data analyzed for frequency. Next, the MPU 52 compares the pulse wave detection data analyzed for frequency and the motion detection data analyzed for frequency, and determines the difference between their frequency components to create differential data. The frequency analysis results of the differential data thus obtained constitute data in which, for example, motion components originating in the deformation of the wrist (increase and decrease in wrist diameter) due to the clenching and unclenching of the hand are substantially removed from the output signal (pulse wave components+motion components) of the pulse wave sensor, that is, pulse wave data that primarily correspond to the pulse wave components. Furthermore, the MPU 52 calculates the pulse rate from the frequency on the assumption that the maximum frequency components from the resulting pulse wave data constitute the pulse spectrum. The MPU 52 then displays the pulse rate on the display device 55.

As described above, according to the first modification of the first embodiment, it is possible to detect and to register accurately motion components generated from deformations in the mounting area typified by deformations in the wrist (increase and decrease in wrist diameter) due to the clenching and unclenching of the hand. Therefore, the motion components can be accurately removed, making it possible to detect accurately pulse wave components, and hence to measure accurately the pulse rate.

### Second Modification of the First Embodiment

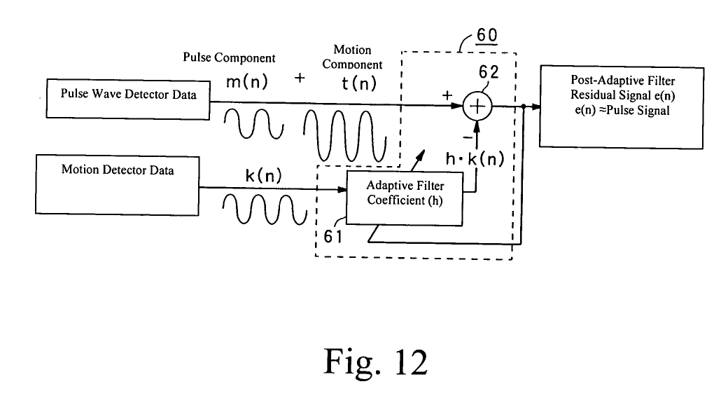

The first embodiment and the first modification of the first embodiment described above use a configuration wherein differential data are calculated by subtracting motion detection data from pulse wave detection data either prior to or after performing frequency analysis (FFT) as an internal process of the MPU 52, but, as shown in FIG. 12, the second modification is one in which motion components are removed from the pulse wave detection data by using an adaptive filter 60. Therefore, the configuration of the second modification of the first embodiment is similar to the configuration of the first embodiment except that the MPU 52 is configured with an adaptive filter 60.

FIG. 12 is a view of a schematic structural block diagram of one example of an adaptive filter. In general terms, the adaptive filter 60 has a filter coefficient generator 61 and a synthesizer 62. The filter coefficient generator 61 functions as a motion component remover and creates an adaptive filter coefficient h on the basis of data previously outputted by the synthesizer 62 to which the filter has been applied. The adaptive filter coefficient h is then applied to the motion detection data (=k(n)), which functions as the inputted motion component detection signal; motion removal data (=h·k(n)) is generated; and these data are outputted to the synthesizer 62. The synthesizer 62 functions as a removal processor, combines the previously extracted pulse wave detection data (=pulse wave components+motion components) and the motion removal data, substantially removes (subtracts) the motion components included in the current pulse wave detection data, and extracts pulse wave components.

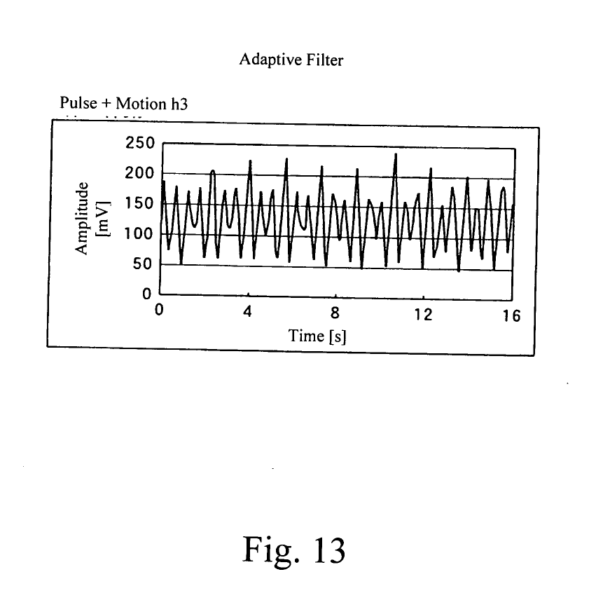

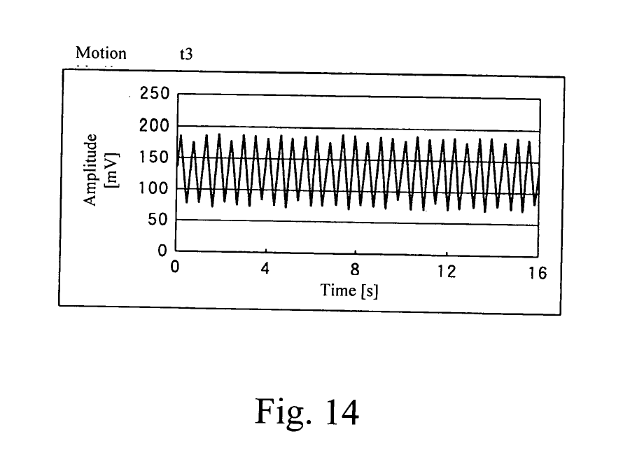

A more specific pulse rate calculation process according to the second modification of the first embodiment will now be described. FIG. 13 is a graph of a chronological arrangement of one example of pulse wave detection data. FIG. 14 is a graph in which motion detection data correlated with the pulse wave detection data in FIG. 13 is chronologically arranged along the same time axis.

First, the MPU 52 sequentially reads the pulse wave detection data and the motion detection data stored in the RAM 53, and outputs the pulse wave detection data in a certain sampling period to the synthesizer 62. In addition, the MPU 52 presents the filter coefficient generator 61 with pressure detection data that correspond to the pulse wave detection data. The filter coefficient generator 31 thereby creates an adaptive filter coefficient h on the basis of the data previously outputted from the synthesizer 62 to which the adaptive filter has been applied. The adaptive filter coefficient h is then applied to the pressure detection data (=k(n)) functioning as the inputted motion component detection signal, and motion removal data (=h·k(n)) is outputted to the synthesizer 62. Thus, the synthesizer 62 combines the current pulse wave data and the motion removal data, substantially removes (subtracts) the motion components included in the current pulse wave detection data, extracts the pulse wave components, and outputs the differential data (=data to which the filter have been applied).

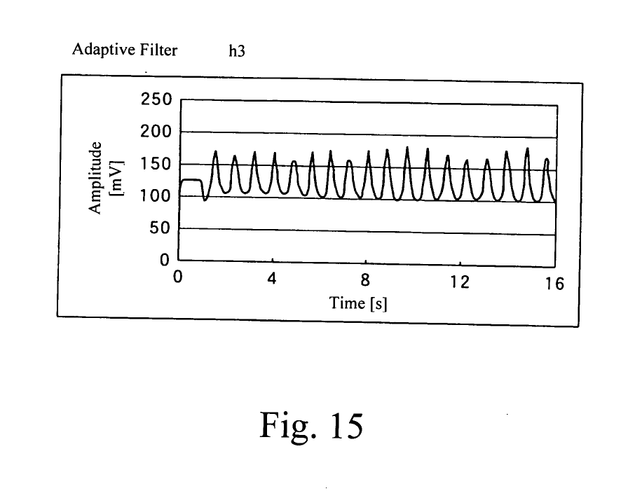

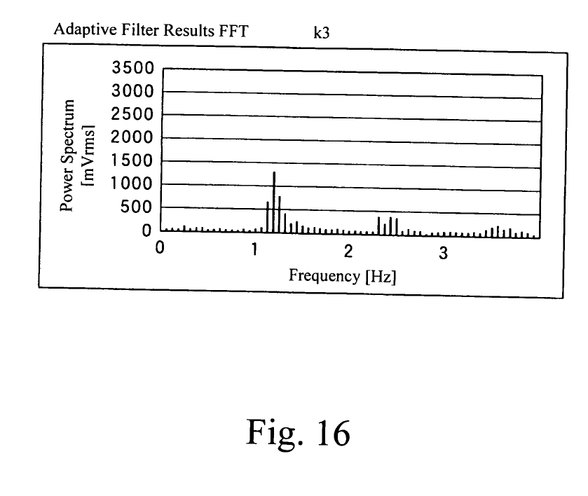

FIG. 15 is a graph of a chronological arrangement of differential data obtained by applying an adaptive filter to the pulse wave detection data in FIG. 13 and the motion detection data in FIG. 14. Next, the MPU 52 subjects the differential data to FFT. FIG. 16 shows the frequency analysis results obtained by subjecting the differential data in FIG. 15 to FFT. The frequency analysis results thus obtained constitute data in which motion components generated from deformations in the mounting area typified by deformations in the wrist (increase and decrease in wrist diameter) due to the clenching and unclenching of the hand are substantially removed from the output signal (pulse wave component+motion components) of the pulse wave sensor, that is, pulse wave data that primarily correspond to the pulse wave components. Furthermore, the MPU 52 calculates the pulse rate from the frequency on the assumption that the maximum frequency components from the resulting pulse wave data that primarily contain pulse wave components constitute the pulse spectrum. The MPU 52 then displays the pulse rate on the display device 55.

As described above, according to the second modification of the first embodiment, it is possible to detect and to register accurately motion components generated from deformations in the mounting area typified by deformations in the wrist (increase and decrease in wrist diameter) due to the clenching and unclenching of the hand. Therefore, the motion components can be accurately removed, making it possible to detect accurately pulse wave components, and hence to measure accurately the pulse rate.

### Second Embodiment

A second preferred embodiment will now be explained. In view of the similarity between the first and second embodiments, the parts of the second embodiment that are identical or substantially identical to the parts of the first embodiment will be given the same reference numerals as the parts of the first embodiment. Moreover, the descriptions of the parts of the second embodiment that are identical to the parts of the first embodiment may be omitted for the sake of brevity.

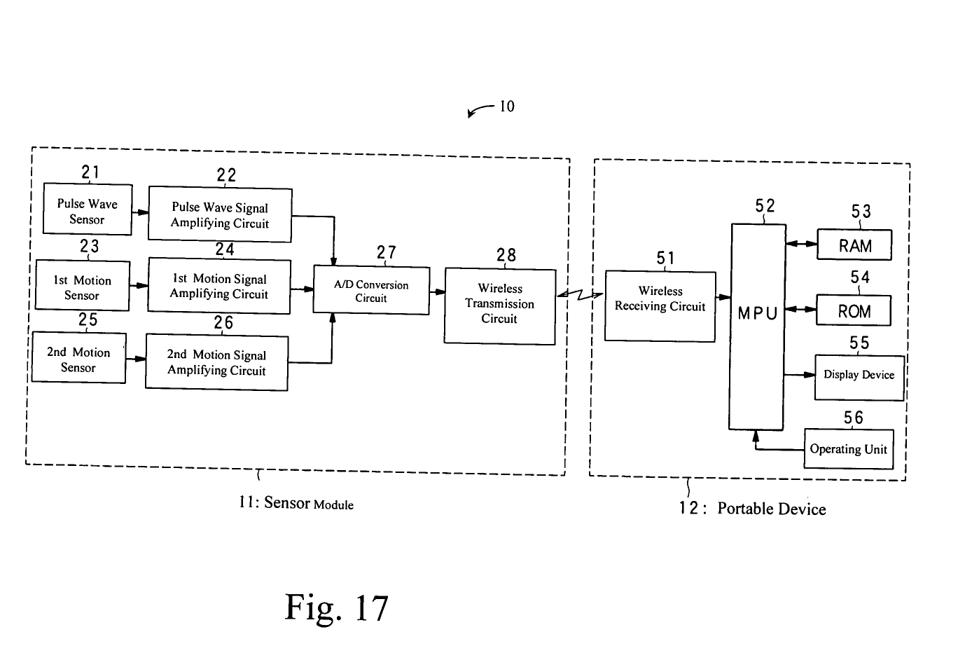

The second embodiment of the present invention will be described with reference to FIG. 17 through FIG. 19. In addition to the configuration of the first embodiment, the second embodiment further includes a sensor to detect motion components generated along with the swinging of the user's arm and other such arm movements, whereby motion components originating in the movements of the arm of the user are removed in addition to motion components originating in changes in the wrist diameter due to the clenching and unclenching of the hand. Therefore, the sensor makes it possible to detect more accurately pulse waves. FIG. 17 is a schematic structural block diagram of a sensor module 11X and a portable device 12 of the second embodiment. In view of the similarity between the first embodiment and the second embodiment, the parts in FIG. 17 similar to or the same as those of FIG. 3 of the first embodiment are denoted by the same symbols.

In general terms, the sensor module 11X has a pulse wave sensor 21, a pulse wave signal amplifying circuit 22, a first motion sensor 23, a first motion signal amplifying circuit 24, a second motion sensor 25, a second motion signal amplifying circuit 26, an A/D conversion circuit 27, and a wireless transmission circuit 28.

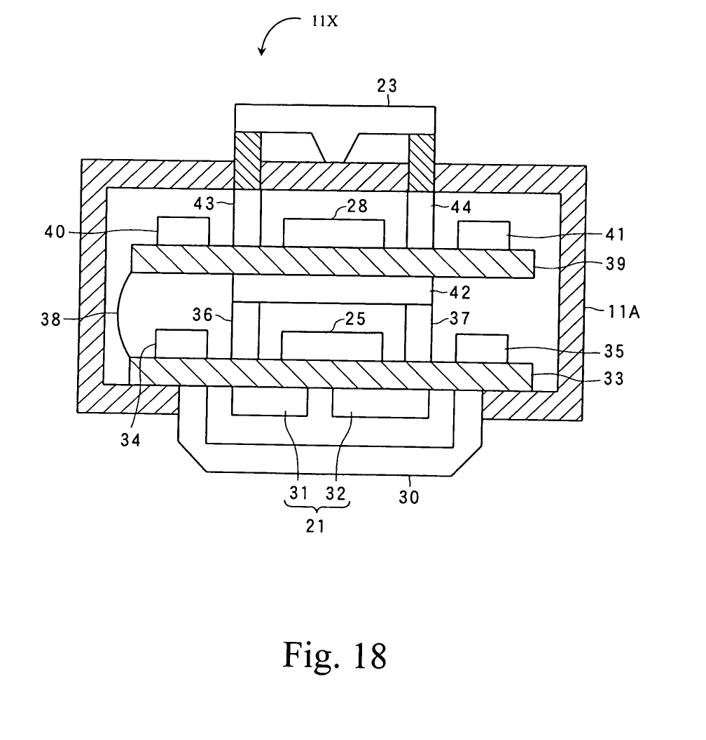

As seen in FIG. 18, the pulse wave sensor 21 has an LED (Light Emitting Diode) 31 and a PD (Photo Detector) 32. Referring again to FIG. 17, further, the pulse wave sensor 21 presents the pulse wave signal amplifying circuit 22 with a pulse wave detection signal that corresponds to the pulsating flow due to the heart rate of blood flowing through the blood vessels. The pulse wave signal amplifying circuit 22 amplifies the inputted pulse wave detection signal at a specific rate of amplification and outputs the result as an amplified pulse wave signal to the A/D conversion circuit 27. The first motion sensor 23 detects changes in the shape of the mounting area of the sensor module 11X, or, specifically, changes in the wrist diameter due to the clenching and unclenching of the hand, and outputs a first motion detection signal to the first motion signal amplifying circuit 24. In this case, the first motion sensor can be configured from a load sensor, a pressure sensor, a displacement sensor, or the like, but an example in which a load sensor is used is described below.

The first motion signal amplifying circuit 24 amplifies the inputted first motion detection signal at a specific rate of amplification, and outputs the result as a first amplified motion signal to the A/D conversion circuit 27. The second motion sensor 25 detects motion components generated along with the swinging of the user's arm and other such arm movements, and outputs a second motion detection signal to the second motion signal amplifying circuit 26. The second motion signal amplifying circuit 26 amplifies the inputted second motion detection signal at a specific rate of amplification, and outputs the result as a second amplified motion signal to the A/D conversion circuit 27.

The A/D conversion circuit 27 performs analog/digital conversion on the inputted amplified pulse wave signal, and outputs the result as pulse wave detection data to the wireless transmission circuit 28. The A/D conversion circuit 27 also performs analog/digital conversion on the amplified first motion signal, and outputs the result as first motion detection data to the wireless transmission circuit 28. The A/D conversion circuit 27 furthermore performs analog/digital conversion on the amplified second motion signal, and outputs the result as second motion detection data to the wireless transmission circuit 28. The wireless transmission circuit 28 modulates the carrier wave on the basis of the inputted pulse wave detection data and the first motion detection data or second motion detection data, and transmits the result to the portable device 12.

FIG. 18 is a schematic cross-sectional view of the sensor module 11X. In view of the similarity between the first embodiment and the second embodiment, the parts in FIG. 18 similar to or the same as those of FIG. 4 of the first embodiment are denoted by the same symbols. The sensor module 11X is designed so that the lower side in FIG. 18 is pressed against the arm of the user. In other words, the cover glass 30 side faces the arm of the user. Therefore, the LED 31 and PD 32 constituting the pulse wave sensor 21 are aligned on a first board 33 in a state protected by the cover glass 30 on the lower side of the casing 11A of the sensor module 11X. The first board 33 is supported by the casing 11A. An acceleration sensor functioning as the second motion sensor 25, circuit elements 34 and 35, and battery supports 36 and 37 are aligned on the upper side of the first board 33. The circuit elements 34 and 35 are circuit elements configured to connect the circuits 22 through 27.

A second board 39 is connected to the first board 33 via a flexible wiring board 38. This second board 39 is supported by the casing 11A. The wireless transmission circuit 28 and circuit elements 40 and 41 are aligned on the upper side of the second board 39. Furthermore, a power source 42 is pressed against the board while supported by the battery supports 36 and 37. Also, the first motion sensor 23 is supported on the upper side of the casing 11A, and the first motion sensor 23 is electrically connected to the second board 39 via conductive members 43 and 44.

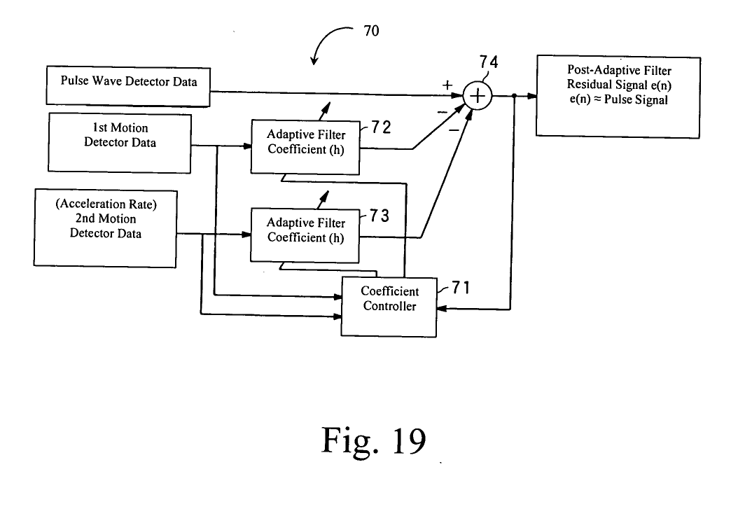

In the second embodiment, as shown in FIG. 19, motion components are removed from pulse wave detection data by using an adaptive filter 70. FIG. 19 is a view of a schematic structural block diagram of one example of the adaptive filter 70. In general terms, the adaptive filter 70 has a filter coefficient controller 71, a first adaptive filter coefficient generator 72, a second adaptive filter coefficient generator 73, and a synthesizer 74.

The filter coefficient controller 71, the first adaptive filter coefficient generator 72 and the second adaptive filter coefficient generator 73 herein function as motion component removers. The filter coefficient controller 71 creates an adaptive filter coefficient h on the basis of data previously outputted by the synthesizer 74 to which the filter has been applied. Further, the adaptive filter coefficient h is outputted to the first adaptive filter coefficient generator 72 and the second adaptive filter coefficient generator 73. Thus, the first adaptive filter coefficient generator 72 applies the adaptive filter coefficient h to the first motion detection data obtained by an A/D conversion of the motion detection signal (first motion detection signal) outputted by the motion sensor 23, then creates first motion removal data, and outputs the result to the synthesizer 74. In addition, the second adaptive filter coefficient generator 73 applies the adaptive filter coefficient h to the second motion detection data obtained by an A/D conversion of the motion detection signal (second motion detection signal) outputted by the motion sensor 25, then creates second motion removal data, and outputs the result to the synthesizer 74.

The synthesizer 74 functions as a removal processor. The synthesizer 74 combines the pulse wave detection data (=pulse wave components+motion components), the first motion removal data, and the second motion removal data, substantially removes (subtracts) the motion components included in the current pulse wave detection data, and extracts pulse wave components. The pulse rate is then calculated and displayed by the same processes as in the second modification of the first embodiment.

Therefore, the second embodiment has a first motion sensor 23 to detect primarily changes in the wrist diameter due to the clenching and unclenching of the hand, and a second motion sensor 25 to detect primarily motion components generated along with the swinging of the user's arm and other such arm movements. Further, a configuration is used wherein an adaptive filter coefficient is applied to first and second motion detection data obtained based on the signals outputted from these sensors, first and second motion removal data are created, and the motion components included in pulse wave detection data are substantially removed, which makes it possible to detect more accurately pulse waves.

### First Modification of Second Embodiment

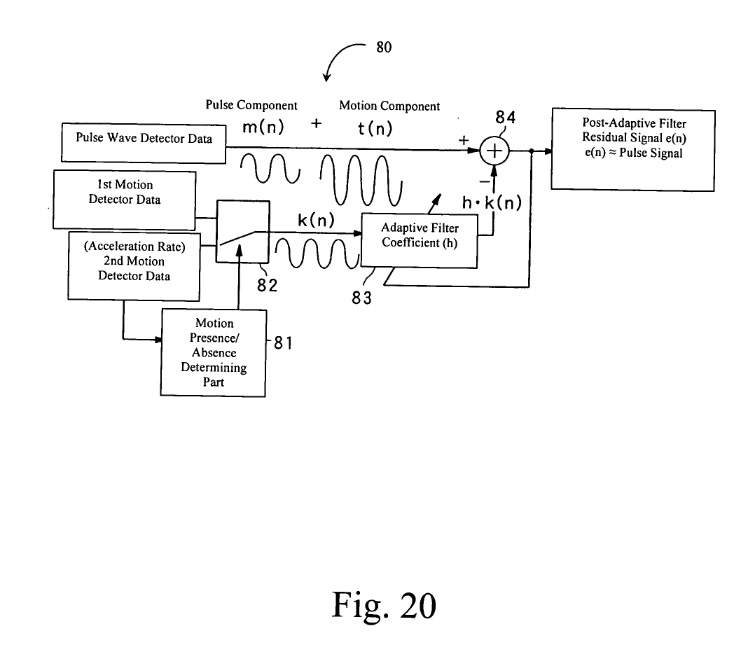

The first modification of the second embodiment will now be described with reference to FIG. 20. The second embodiment described above involves extracting pulse wave components by using all of the pulse wave detection data (=pulse wave components+motion components), the first motion detection data, and the second motion detection data. In comparison, the first modification of the second embodiment is one wherein the first motion detection data corresponding to motion components originating in changes in the shape of the mounting area have a marked effect during rest and a small effect during movement (walking, running), and wherein, conversely, the second motion detection data have a small effect during rest and a marked effect during movement (walking, running). In other words, pulse wave components are extracted using pulse wave detection data and first motion detection data in the absence of considerable motion, or, specifically, during rest. Conversely, pulse wave components are extracted using pulse wave detection data and second motion detection data during considerable motion, or, specifically, during movement. Therefore, the device configuration and processing are simplified because only one adaptive filter coefficient generator need be provided. Therefore, the configuration of the first modification of the second embodiment is similar to that of the second embodiment except that the MPU 52 is configured to have an adaptive filter 80 instead of the adaptive filter 70 in the second embodiment, so descriptions of parts similar to or the same as those of the second embodiment are omitted for the sake of simplicity.

FIG. 20 is a view of a schematic structural block diagram of one example of the adaptive filter 80. In general terms, the adaptive filter 80 has a motion presence/absence determining section 81, a data switcher 82, a filter coefficient generator 83, and a synthesizer 84. The motion presence/absence determining section 81 distinguishes whether there is considerable motion on the basis of the second motion detection data, and outputs a switching signal to the data switcher 82. As a result, the data switcher 82 switches to first motion detection data when it is determined that there is no considerable motion. Therefore, the filter coefficient generator 83 creates an adaptive filter coefficient h on the basis of data outputted previously by the synthesizer 84 to which the filter has been applied. The adaptive filter coefficient h is then applied to the first motion detection data (=k(n)), which functions as an inputted motion component detection signal; first motion removal data (=h·k(n)) are generated; and these data are outputted to the synthesizer 84. The synthesizer 84 functions as a removal processor, combines the previously extracted pulse wave detection data (=pulse wave components+motion components) and the first motion removal data, substantially removes (subtracts) the motion components included in the current pulse wave detection data, and extracts pulse wave components.

Conversely, the data switcher 82 switches to second motion detection data due to the switching signal when the motion presence/absence determining section 81 determines that there is considerable motion. Therefore, the filter coefficient generator 83 creates an adaptive filter coefficient h on the basis of data outputted previously by the synthesizer 84 to which the filter has been applied. The adaptive filter coefficient h is then applied to the second motion detection data (=k(n)), which functions as the inputted motion component detection signal; second motion removal data (=h·k(n)) are generated; and these data are outputted to the synthesizer 84. The synthesizer 84 functions as a removal processor, combines the previously extracted pulse wave detection data (=pulse wave components+motion components) and the second motion removal data, substantially removes (subtracts) the motion components included in the current pulse wave detection data, and extracts pulse wave components.

According to the first modification of the second embodiment as described above, it is possible to simplify the device configuration, to make processing less complicated, and to extract accurately pulse wave components. As a result, the pulse rate can be accurately detected.

### Application of the Invention

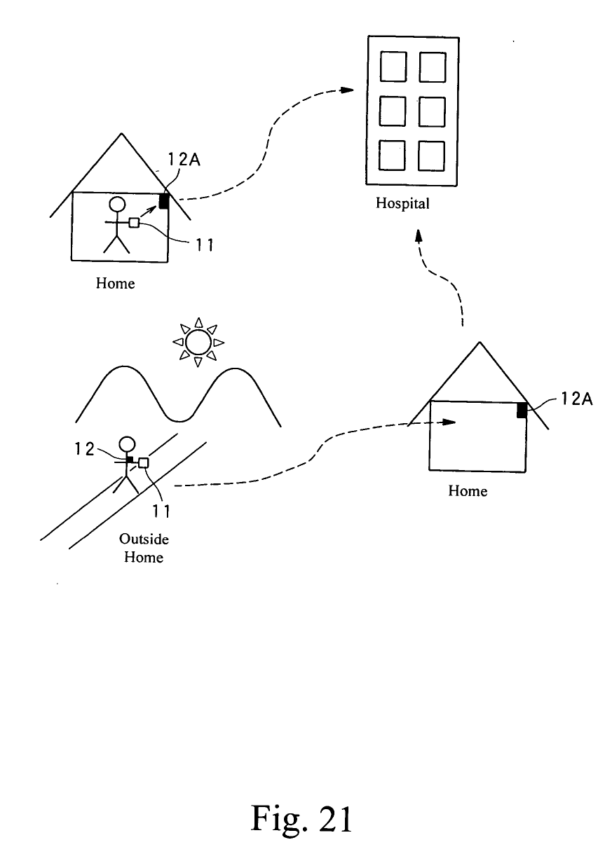

An application of the pulse measurement system of the present invention will now be described. FIG. 21 is a view of an explanatory diagram of an application of the pulse measurement system of the present invention. As shown in FIG. 21, when the user is at home, the sensor module 11 is mounted on the arm, the configuration used in the home is the same as that of the portable device 12, and a stationary device 12A, which is connected via a phone line or another such network to a hospital or the like (the destination of the pulse rate data), is left in an operating state. Thus, the pulse wave detection data and the motion detection data detected by the sensor module 11 are received via the wireless receiving circuit of the stationary device 12A by the wireless transmission circuit 28, and is communicated to the hospital via the network. The stationary device 12A essentially fulfills the same function as the portable device 12, except that it includes a configuration that enables a connection with a hospital or the like via a phone line or other such network.

Also, the user mounts the sensor module 11 on the arm and carries the portable device 12 when going outside. The pulse wave detection data and the motion detection data detected by the sensor module 11 are thereby received via the wireless receiving circuit 51 of the portable device 12 by the wireless transmission circuit 28, and the pulse rate data is stored in the RAM 53. The pulse rate data can subsequently be communicated to the hospital via a phone line or another such network by connecting the portable device 12 to the stationary device 12A via a communication interface (not shown).

### Modification When Power Generating Device is Used

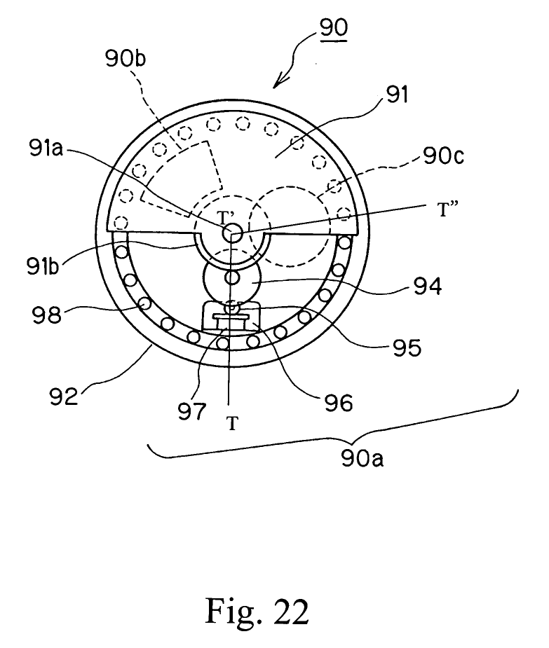

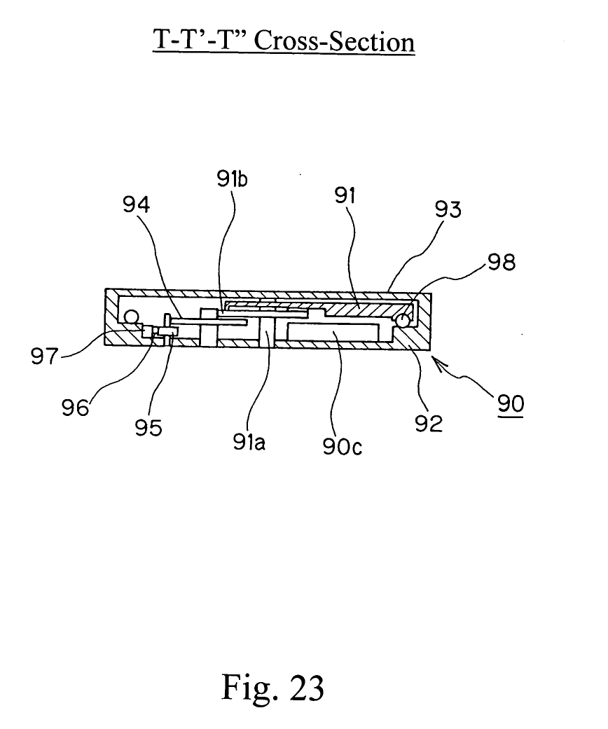

In the above descriptions, a case in which a battery is used as the power source of the sensor module was described, but it is also possible to use a compact power generation device instead of the battery. FIG. 22 is an elevational view showing the configuration of a power generation device 90, and FIG. 23 is a schematic cross-sectional side view of the power generation device 90 in FIG. 22. The power generation device 90 is configured from a power generating mechanism 90a, a voltage control circuit 90b, and a capacitor 90c. The power generating mechanism 90a is configured to generate power by the rotation of a rotary spindle 91 due to movements of the hand of the user and the like.

Specifically, as shown in FIGS. 22 and 23, the power generating mechanism 90a has a case that includes a base 92 and a cover 93. Further, the rotary spindle 91, which rotates around a rotating shaft 91a fixed to the base 92, is mounted inside the case. The rotary spindle 91 is shaped such that the center of gravity thereof is significantly misaligned from the position of the rotating shaft 91a. Furthermore, a gear 91b is fixed to the rotary spindle 91, and the gear 91b is designed to rotate in accordance with the rotation of the rotary spindle 91.

Also, a middle gear 94 that rotates in accordance with the rotation of the gear 91b, and a power-generating rotor 95 that rotates in accordance with the rotation of this middle gear, are provided inside the above-mentioned case. The gear 91b and middle gear 94 form a rotary movement transmission mechanism, commonly referred to as a gear train mechanism.

The power-generating rotor 95 is formed from the rotating shaft thereof and a permanent magnet that is fixed to the rotating shaft and has the N-pole and S-pole in a direction orthogonal to the rotating shaft. Furthermore, a roughly C-shaped stator 96 having highly magnetically permeable material is disposed to hold the power-generating rotor 95 between both ends, and a conductive wire is wound around the central portion of the stator 96 to form a coil 97.

Also, a bearing 98 to support the rotation of the rotary spindle 91 is disposed between the base 92 and the rotary spindle 91. The voltage control circuit 90b and the capacitor 90c are disposed in the open space around the rotating shaft 91a of the base 92.

The power generating mechanism 90a described above generates power as follows. Specifically, when the rotary spindle 91 rotates due to movements of the arm of the user or the like, this rotational movement is transmitted to the power-generating rotor 95 and causes the power-generating rotor 95 to rotate. When the power-generating rotor 95 rotates, the permanent magnet of the power-generating rotor 95 rotates, the both magnetic poles of the permanent magnet alternately face the ends of the stator 96 along with the rotation, and the magnetic flux generated from the N-pole of the permanent magnet at this moment passes through the stator 96 and reaches the S-pole. The magnetic flux is thereby caused to pass instantaneously along the winding axis of the coil 97. The magnetic flux passing through the coil 97 is reversed synchronously with the rotation of the power-generating rotor 95. An induced electromotive force based on Lentz's Law is thereby produced in the coil 97, power is generated, and the AC power is outputted from both ends of the coil 97 along with the rotation of the rotary spindle 91.

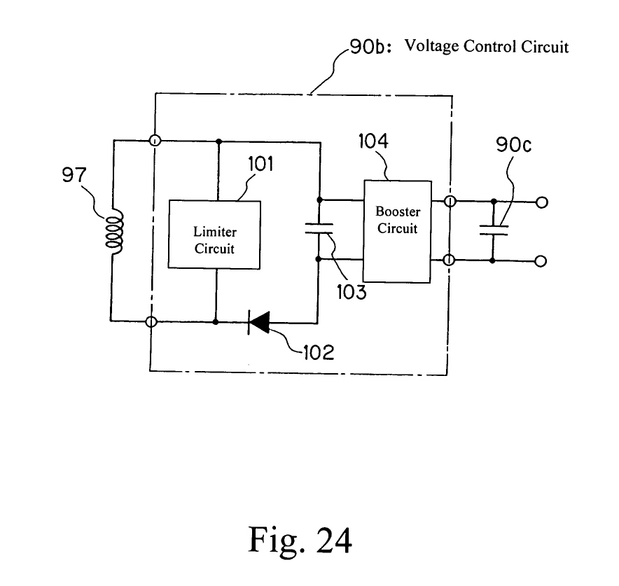

As shown in FIG. 24, the voltage control circuit 90b is configured from a limiter circuit 101, a diode 102, a capacitor 103, and a booster circuit 104. The limiter circuit 101 is connected in parallel with the coil 97, and is designed to prevent induced electric current from being outputted from the coil 97 when a specific upper limit is exceeded. Thus, circuits connected to subsequent stages are prevented from being disrupted or the like even when a large induced electric current is generated.

The diode 102 and the capacitor 103 are connected in series, and this series circuit is connected in parallel with the limiter circuit 101. The induced electric current generated in the coil 97 is rectified by the diode 102, and is temporarily stored in the capacitor 103.

As is conventionally known, the booster circuit 104 outputs the inputted voltage multiplied by a specific rate, and the input side thereof is connected to both ends of the capacitor 103. Thus, the voltage stored in the capacitor 103 is raised by the booster circuit 104 and outputted. The capacitor 90c is connected in parallel with the output side of the booster circuit 104, and the electric power outputted from the booster circuit 104 is stored in the capacitor 90c. In addition, a secondary battery (not shown in the figures) is connected to the capacitor 90c; therefore, the secondary battery is also charged by the output of the booster circuit 104, and the electric energy stored in the capacitor 90c and the secondary battery is supplied as a power source.

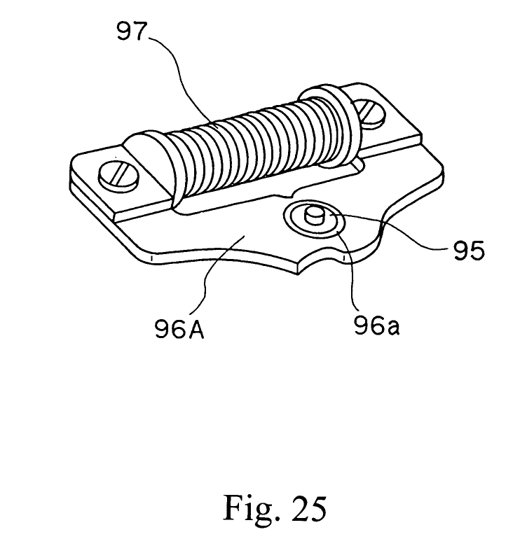

Therefore, since the sensor module 11 is driven by electric power generated by the use of kinetic energy when the module is worn by the user, semi-permanent use is possible and there is no need to exchange batteries as in conventional practice. Also, combined use of the power generation device 90 and the secondary battery in the sensor module 11 makes it possible to exert adequately sensing functions because electric power is supplied even when there is no power generation. Furthermore, since the power generation device 90 charges the secondary battery, it is possible to utilize efficiently the part of the generated electric energy that cannot be consumed by the sensor module. Furthermore, with the power generation device 90, there are no failures due to cracks such as those seen in power generation devices that use ceramic piezoelectric elements in conventional technology, long-term stable power generation is possible, and excellent reliability and durability can be ensured. Moreover, as shown in FIG. 25, a monolithic stator 96A with a roughly circular opening 96a through which the power-generating rotor 95 is inserted may be used instead of the stator 96. Furthermore, employing the above-described configuration in a pitch meter or pedometer eliminates the need to replace batteries and makes it possible to configure a pitch meter or pedometer capable of semi-permanent use.

### Modification of Sensor Module

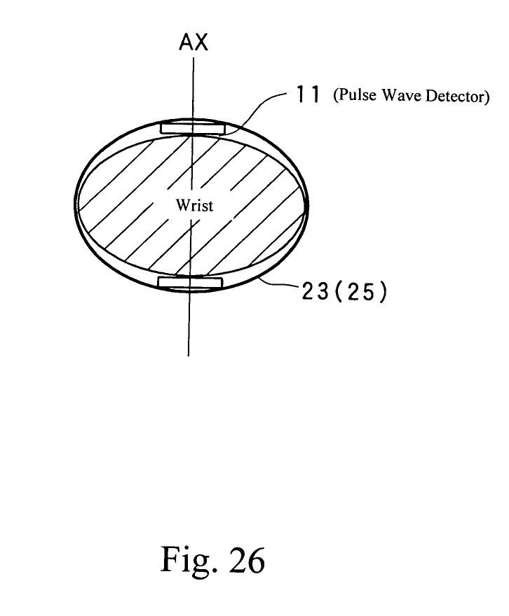

In the above descriptions, a pulse wave detection sensor and a motion detection sensor were mounted in the sensor module 11 as shown in FIG. 2, but, as shown in FIG. 26, it is also possible to use a configuration wherein only a pulse wave detection sensor is mounted in the sensor module 11, and the motion detection sensor 23 (25) is mounted in a symmetric position on the other side of the wrist (mounting area), or, specifically, on the same axis AX on the other side of the wrist.

The above description was given with reference to a case in which a control program was stored in advance in the ROM 310 of a controller 5, but another possibility is a configuration wherein the control program is stored in advance on various magnetic disks, optical disks, memory cards, and other such storage media, and is read from these storage media and installed. Another possibility is a configuration wherein the control program is downloaded via the Internet, LAN, or another such network; and the control program is then installed and run.

This specification claims priority to Japanese Patent Application No. 2003-075838. Japanese Patent Application No. 2003-075838 is incorporated herein by reference.

As used herein, the following directional terms “forward, rearward, above, downward, vertical, horizontal, below, and transverse” as well as any other similar directional terms refer to those directions of an information gathering device and a pulse meter with the present invention. Accordingly, these terms, as utilized to describe the present invention should be interpreted relative to an information-gathering device and a pulse meter equipped with the present invention.

“Front,” “back, ” “up,” “down,” “perpendicular,” “horizontal,” “slanted,” and other terms used hereinabove for indicating direction are convenient terms used for describing the embodiments of the present invention. Therefore, the terms for indicating these directions and used for describing the present invention should be interpreted in relative terms.

The term “configured” as used herein to describe a component, section or part of a device includes hardware and/or software that is constructed and/or programmed to carry out the desired function.

Moreover, terms that are expressed as “means-plus function” in the claims should include any structure that can be utilized to carry out the function of that part of the present invention.

The terms of degree such as “substantially,” “about,” and “approximately” as used herein mean a reasonable amount of deviation of the modified term such that the end result is not significantly changed. For example, these terms can be construed as including a deviation of at least ±5% of the modified term if this deviation would not negate the meaning of the word it modifies.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents. Thus, the scope of the invention is not limited to the disclosed embodiments.

What is claimed is:

1. An information-gathering device being configured to gather information relating to a pulse comprising:
a sensor module being configured to be mounted to a mounting area on a body; and
a supporter being configured to mount said sensor module, said sensor module comprising,
a motion detector being configured to detect motion components generated along with changes in the shape of said mounting area, and to output a motion detection signal, and
a pulse wave detector being configured to detect pulse wave components corresponding to a pulsating flow in said body, and to output a pulse wave detection signal.

2. The information-gathering device according to claim 1, wherein
said motion detector comprises a pressure sensor, a load sensor, or a displacement sensor.

3. The information-gathering device according to claim 1, wherein
said pulse wave detector comprises a pulse wave sensor, and
said motion detector has a detection position thereof disposed adjacent said pulse wave sensor.

4. The information-gathering device according to claim 2, wherein
said pulse wave detector comprises a pulse wave sensor, and
said motion detector has a detection position thereof disposed on a reverse side or same side of said mounting area in relation to said pulse wave sensor, said motion detector is positioned such that the same axis that passes through a detection position of said pulse wave sensor and said mounting portion passes through said motion detector

5. The information-gathering device according to claim 1, wherein said sensor module comprises a transmitter being configured to transmit said motion detection signal and said pulse wave detection signal.

6. The information-gathering device according to claim 5, wherein said sensor module further comprises a power generation device connected to said motion detector and said pulse wave detector configured to convert kinetic energy to electric energy and to supply electric energy to said information-gathering device.

7. A pulse meter, comprising:
a motion detector being configured to detect motion components and changes in a shape of a mounting area of a body, and to output a motion detection signal;
a pulse wave detector being configured to detect pulse wave components corresponding to a pulsating flow in said body, and to output a pulse wave detection signal;
a transmitter being configured to transmit said motion detection signal and said pulse wave detection signal;
a receiver being configured to receive said motion detection signal and said pulse wave detection signal from said transmitter; and
a pulse rate calculator being configured to calculate said pulse rate using said motion detection signal and said pulse wave detection signal received by said receiver.

8. The pulse meter according to claim 7, wherein
said pulse rate calculator comprises a removal processor being configured to subtract said motion detection signal from said pulse wave detection signal.

9. The pulse meter according to claim 7, wherein
said pulse rate calculator comprises,
a first frequency analyzer being configured to perform frequency analysis on said motion detection signal and to generate first frequency analysis data,
a second frequency analyzer being configured to perform frequency analysis on said pulse wave detection signal and to generate second frequency analysis data and
a removal processor being configured to perform subtraction processing on said first frequency analysis data corresponding to said second frequency analysis data.

10. The pulse meter according to claim 7, wherein
said pulse rate calculator comprises,
a filter coefficient generator being configured to generate an adaptive filter coefficient using said pulse wave detection signal and said motion detection signal, and
a removal processor being configured to subtract from said pulse wave detection signal said motion component detection signal to which said adaptive filter coefficient has been applied.

11. An information-gathering device being configured to gather information relating to a pulse comprising:
a sensor module being configured to be mounted to a mounting area on a body; and
a supporter being configured to mount said sensor module, said sensor module comprising,
a first motion detector being configured to detect motion components generated along with changes in the shape of said mounting area, and to output a first motion detection signal,
a second motion detector being configured to detect motion components generated along with body movement, and to output a second motion detection signal, and
a pulse wave detector being configured to detect pulse wave components corresponding to a pulsating flow in said body, and to output a pulse wave detection signal.

12. The information-gathering device according to claim 11, wherein said sensor module comprises a removal processor being configured to remove motion components using said second motion detection signal when said second motion detector detects said motion components, and to remove motion components using said first motion detection signal when said second motion detector does not detect said motion components.

13. The information-gathering device according to claim 11, wherein
said first motion detector comprises a pressure sensor, a load sensor, or a displacement sensor.

14. The information-gathering device according to claim 11, wherein
said second motion detector comprises an acceleration sensor.

15. The information-gathering device according to claim 11, wherein
said pulse wave detector comprises a pulse wave sensor, and
said first and second motion detectors have detection positions thereof disposed adjacent said pulse wave sensor.

16. The information-gathering device according to claim 11, wherein
said pulse wave detector comprises a pulse wave sensor, and
said first and second motion detectors have the detection positions thereof disposed on the reverse side or the same side of said mounting area in relation to said pulse wave sensor, said first and second motion detectors are positioned such that the same axis that passes through a detection position of said pulse wave sensor passes through said first and second motion detectors.

17. The information-gathering device according to claim 11, wherein said sensor module comprises a transmitter being configured to transmit said motion detection signal and said pulse wave detection signal.

18. The information-gathering device according to claim 17, further comprising a power generation device connected to said first and second motion detectors and said pulse wave detector to supply electric energy.

19. The information-gathering device according to claim 17, further comprising,
a portable device having a receiver, and a pulse rate calculator,
said receiver being configured to receive said first and second motion detection signals and said pulse wave detection signal from said transmitter and
said pulse rate calculator being configured to calculate said pulse rate using said first and second motion detection signals and said pulse wave detection signal received by said receiver.

20. The information-gathering device according to claim 19, wherein
said pulse rate calculator comprises,
a filter coefficient generator being configured to generate an adaptive filter coefficient using said pulse wave detection signal and at least one of said first motion detection signal and said second motion detection signal, and
a removal processor being configured to subtract from the pulse wave detection signal at least one of said first motion detection signal to which said adaptive filter coefficient has been applied and said second motion detection signal to which said adaptive filter coefficient has been applied.

21. A method for gathering information relating to the pulse comprising:
mounting on a body an information-gathering device on a mounting area;
detecting motion components of said mounting area;
detecting changes in shape of said mounting area;
outputting a motion detection signal;
detecting pulse wave components corresponding to a pulsating flow in said body;
outputting a pulse wave detection signal; and
calculating a pulse rate using said motion detection signal and said pulse wave detection signal.

22. A control program for gathering information relating to the pulse from a mounting area on the body, comprising:
code for detecting motion components of the mounting area;
code for detecting changes in shape of the mounting area;
code for receiving a motion detection signal;
code for detecting pulse wave components corresponding to a pulsating flow in the body;
code for receiving a pulse wave detection signal; and
code for calculating the pulse rate using the motion detection signal and the pulse wave detection signal.

23. A computer-readable storage medium comprising:
a control program being stored in said storage medium and designed to gather information relating to a pulse from a mounting area on a body said control program including commands for,
code for detecting motion components generated during movement of said mounting area,
code for detecting motion components generated during changes in the shape of said mounting area,
code for receiving a motion detection signal,
code for detecting pulse wave components corresponding to a pulsating flow in the body,
code for receiving a pulse wave detection signal, and
code for calculating the pulse rate using said motion detection signal and the pulse wave detection signal.
